(19) United States
(12) Patent Application Publication
(10) Pub. No.: US 2011/0027352 A1
(43) Pub. Date: Feb. 3, 2011

(54) COMPOUNDS FOR USE IN THE TREATMENT OF NEUROPATHIC PAIN

(75) Inventors: Michel Barrot, Strasbourg (FR); Ipek Yalcin, Bischheim (FR); Marie-José Freund-Mercier, Strasbourg (FR); Malika Benbouzid, Strasbourg (FR); Nada Choucair-Jaafar, Strasbourg (FR)

Correspondence Address: ARENT FOX LLP, 1050 CONNECTICUT AVENUE, N.W., SUITE 400, WASHINGTON, DC, 20036, US

(73) Assignee: CENTRE NATIONAL DE LA RECHERCHE SCIENTIFIQUE - CINRS, Paris Cedex 16 (FR)

(21) Appl. No.: 12/863,380

(22) PCT Filed: Jan. 16, 2009
(86) PCT No.: PCT/FR09/00045

§ 371 Date: Oct. 19, 2010

(30) Foreign Application Priority Data
Jan. 18, 2008 (FR) 0800273
Feb. 26, 2008 (EP) 08003458.0
Nov. 14, 2008 (EP) 08291065.4

(51) Int. Cl. A61K 31/137; A61K 31/27; A61K 31/167; A61K 31/138; A61K 9/28; A61K 9/48; A61P 25/00

(52) U.S. Cl. 424/451; 514/653; 514/483; 514/629; 514/651; 424/474

(57) ABSTRACT

The present invention relates to the use of a beta-adrenergic receptor agonist as active ingredient for the production of a medicament for use in the treatment of neuropathic pain, in particular neuropathic allodynia, in particular chronic neuropathic allodynia, and more generally for the production of medicaments for relieving pain. The principal field of application of the present invention is the biomedical field, and more specifically the therapeutics field. The present invention aims in particular to provide a medicament which can be used as a substitute for the antidepressants currently used to treat pain. It finds a use in the human and veterinary clinical field.

### TECHNICAL FIELD OF THE INVENTION

The present invention relates to the use of compounds which are beta-adrenergic receptor agonists, as active ingredients, for the production of medicaments for use in the treatment of neuropathic pain, in particular neuropathic allodynia, in particular chronic neuropathic allodynia, and more generally for the production of medicaments for use in pain relief.

The principal field of application of the present invention is the biomedical field, and more specifically the therapeutics field. It finds an application in the human and veterinary clinical field.

The present invention aims in particular to provide medicaments which can be used as a substitute for the treatments currently used to treat pain.

In the description which follows, the references between parentheses (x) refer back to the list of references located at the end of the examples.

### PRIOR ART

According to a recent clinical study (Bouhassira et al., Pain, 2008, 136:380-387 (1)), the prevalence of chronic pain with a neuropathic component is 6.9% in the general population in France, thereby affecting approximately 4 million individuals in France.

Neuropathic pain is a generally chronic disease linked to a nervous system dysfunction or lesion as described in Merksey and Bogduk (1994) (2). It involves long-lasting molecular modifications. Effective long-term treatments also involve plasticity of the nervous system: they are ineffective in the first days, and then relieve pain.

Chronic neuropathic pain is difficult to treat clinically since most of the conventional analgesic treatments are ineffective over time. This is in particular the case of opiates, for which tolerance to the effects develops.

The pharmacological treatment of persistent neuropathic pain currently mainly uses either anticonvulsants, for example pregabalin or gabapentin; or tricyclic antidepressants, for example amitriptylin, nortriptylin, clomipramin, imipramin or desipramin, or mixed serotonin and noradrenalin reuptake inhibitor antidepressants, for example duloxetin or venlafaxi. Currently, anticonvulsants and antidepressants are the two prescribed options recommended in various countries as first line treatment for combating neuropathic pain (McQuay et al., 1996, Pain 68:217-227 (3); Hempenstall et al. PLoS Med, 2005, 2:e164 (4); Gilron et al., CMAJ, 2006, 175:265-275 (5); Attal et al., European Journal of Neurology, 2006, 13: 1153-1169 (6); Moulin et al., Pain Research Management, 2007, 12:13-21 (7); Dworkin et al., Pain, 2007, 132:237-251 (8)).

As for most chronic diseases affecting the nervous system, no available medicament is both effective in and tolerated by all patients.

For example, while antidepressants are effective, their use in fact poses certain problems:
1) Psychological effects: some patients suffering from pain have trouble accepting the prescription of an “antidepressant” known to treat psychiatric disorders, although they are not depressive;
2) side effects which may be poorly tolerated and which affect approximately 30% of patients (McQuay et al., 1996 (3)): for example, drowsiness or sedation, blurred vision, constipation, urinary retention or dysuria, a dry mouth, hyperhidrosis, weight gain, tachycardia or a cardiac arythmia, orthostatic hypotension, impotence, shaking, convulsions, mental confusion, an increase in aggressiveness, paranoia and suicidal behavior, an increase in breast volume, galactorrhea, allergic skin reactions, dysarthria, eosinophilia, leucopenia, agranulocytosis, thrombocytopenia, syncopy;
3) a lack of specificity of action: the antidepressant, by increasing extracellular levels of monoamines (such as adrenalin, noradrenalin and serotonin), acts on all adrenoreceptors and also on serotonergic receptors, whether or not these various receptors have an effect on neuropathic pain;
4) a variability in action between patients: antidepressants are effective on only a fraction of patients (between 30% and 50% of patients) (McQuay, 1996 (3); Attal et al., 2006 (6)).

It is essential for the clinician to have a range of drugs that is more diversified than that which is currently known, in order to propose to the patient the one which is most suitable for his or her case.

There is therefore a real need for new molecules that can be used in the treatment of pain and that are more effective, better tolerated, and more readily accepted and have a more targeted action.

Other treatments for neuropathic pain are currently used, for example neurostimulation techniques, surgical techniques, acupuncture techniques and psychoanalysis. However, these techniques require repeated intervention by the practitioner(s) and optionally hospitalization; they are difficult to implement, lengthy and/or not always satisfactory.

### DESCRIPTION OF THE INVENTION

The present invention actually meets this need and solves the drawbacks of the prior art.

During the more than 30 years that antidepressants have been used clinically for relieving neuropathic pain, it has never yet been envisioned that beta-adrenergic receptor agonists might have the same effect and might be used as substitutes for antidepressants for long-lasting relief of neuropathic pain. This situation doubtless comes as a result of the poor knowledge of the precise mechanism of action of antidepressants on pain, that was assumed to be related to alpha2-adrenoreceptors. This is because, when taken acutely, alpha2-adrenoreceptor agonists can facilitate analgesia and are used routinely as combined therapy when treating perioperative pain (Crassous et al., 2007, Current Topics in Medicinal Chemistry 7:187-194 (9)). It therefore appeared to be simple and natural to imagine that they were also responsible for the effect of antidepressants, although this has never been proved. We have demonstrated that this was not the case and that, in reality, the adrenoreceptors essential for the therapeutic effect of antidepressants on neuropathic pain are the beta2-adrenoreceptors.

The inventors of the present have identified the adrenoreceptors responsible for the therapeutic effect of antidepressants against chronic neuropathic pain. They are the adrenoreceptors of the beta family, and in particular those of the beta2 subtype. In a murine model of neuropathic pain, they have in fact caused the therapeutic action of antidepressants to disappear by blocking beta or beta2 adrenoreceptors. In addition, the administration only of various beta2-adrenergic or beta-adrenergic agonists is sufficient to relieve neuropathic allodynia in this model. Allodynia, which is a painful sensation in response to a stimulus which should not be painful, is an important symptom of neuropathic pain. Beta2-adrenergic agonists, and more broadly beta-adrenergic agonists, may therefore make it possible to treat neuropathic pain and offer a more targeted alternative to the current use of antidepressants.

Thus, the present invention relates to the use of a beta-adrenergic agonist as an active ingredient for the production of a drug for use in the treatment of neuropathic pain, in particular in the treatment of neuropathic allodynia. According to the invention, these forms of pain may be chronic.

The term “neuropathic pain” is intended to mean pain related to a dysfunction and/or to a lesion of any one of the constitutive elements of the nociceptive pathways as described in Merksey and Bogduk (1994) (2). This comprises, for example, pain subsequent to lesions or irritations of peripheral nerves (whether there is axon and/or myelin involvement), of the spinal cord, or of superspinal structures. The lesion or the dysfunction may, for example, be of mechanical origin, for example post-traumatic; post-surgical; for example related to a nerve lesion under a scar, as is the case in a post-thoracotomy syndrome; to phantom limb in the case of a limb amputation; to a nerve compression in entrapment neuropathies; to a radicular compression in a radiculopathy caused by herniated disc, a situation which can be accompanied by an inflammatory component; to a section or a trauma of the spinal cord causing pain in paraplegic patients; for example of metabolic origin, for example neuropathy due to alcoholism or to diabetes, dysthyroidism; deficiency neuropathies; for example of ischemic origin, for example related to a peripheral arterial ischemia, a spinal ischemia, a stroke; for example of toxic origin, for example related to drugs, for example anticancer chemotherapies based on periwinkle derivatives, on platinum salts; to amiodarome; to industrial toxic substances, for example acrylamide, adhesives, etc.; for example of infectious origin, for example HIV, herpes, chickenpox, shingles; for example of immunoallergic origin, for example paraneoplastic neuropathies, Guillain-Barré syndrome; for example of hereditary origin, for example small fiber neuropathies.

According to the invention, the beta-adrenergic agonist is preferably a beta2-adrenergic agonist or a less specific molecule having, inter alia, a beta2-adrenergic agonist action.

According to the invention, the beta-adrenergic agonist may be chosen, for example, from the group comprising bambuterol, bitolterol, clenbuterol, fenoterol, formoterol, isoproterenol (or isoprenalin), levalbuterol, metaproterenol, pirbuterol, procaterol, reproterol, ritodrin, salbutamol (or albuterol), salmeterol, terbutalin or tulobuterol. The term “agonist” is also hereby intended to mean the pharmaceutically acceptable salts of the abovementioned agonists and/or mixtures of these agonists and/or of the pharmaceutically acceptable salts thereof.

For the purpose of the present invention, pharmaceutically acceptable salts are, for example, hydrochlorides, sulfates, bromides, hydrobromides, hydrates (for example, dihydrates, trihydrates, tetrahydrates, and the like), sodium salts, fumarates, tartrates, mesylates, nitrates, dinitrates, maleates, acetates, citrates, propionates, xinafoates or hydroxynafoates.

According to the invention, the drug may be for human or veterinary use.

According to the invention, the medicament may be in any form suitable for it to be able to be administered to a patient or to an animal. The administration may be carried out directly, i.e. using the pure or substantially pure agonist or a pharmaceutically acceptable salt of the agonist which is pure or substantially pure, or a mixture with a pharmaceutically acceptable carrier or in a pharmaceutically acceptable medium. The medicament may therefore be the agonist, or a salt thereof, in pure or substantially pure form, or the agonist in a suitable solvent or else in a galenic form suitable for its administration to a patient or an animal and for its absorption by a patient or an animal.

The medicine may, for example, be in a form chosen from the group comprising an injectable form (for example, Ventolin (registered trademark)), a syrup (for example, Atarax (registered trademark)), an oral solution (for example, Efferalgan (registered trademark) 3%), a tablet (for example, Aspirine du Rhone (registered trademark) [aspirin]), a breakable tablet (for example, Claradol (registered trademark)), a film-coated tablet (for example, Apranax (registered trademark)), a breakable film-coated tablet (for example, Zyrtecset (registered trademark)), a gastro-resistant tablet (for example, Aspirin (registered trademark) PH 8500 mg gastroresist tab), a coated tablet (for example, Fervex (registered trademark)), a dispersible tablet (for example, Spasmocalm (registered trademark)), a chewable tablet (for example, Rennie (registered trademark)), a blister pack (for example, Ventodisks (registered trademark)), a gel capsule (for example, Polyprine (registered trademark) gel), an effervescent product (for example, Efferalgan (registered trademark)), a breakable effervescent tablet (for example, Prednisolone Arrow (registered trademark)), a powder for oral solution (for example, Doliprane (registered trademark)), granules for oral suspension (for example, Apranax (registered trademark)), a suspension for inhalation (for example, Airomir Autohaler (registered trademark)), a powder for inhalation (for example, Asmelor Novalizer (registered trademark)), a solution for inhalation by nebulizer (for example, Salbutamol Arrow (registered trademark)), a powder for inhalation in a gel capsule (for example, Foradil (registered trademark)), a suppository (for example, Doliprane (registered trademark)), an eye lotion (for example, Rifamycin Chibret (registered trademark)), a cream (for example, Fucidin (registered trademark) 2%), an ointment (for example, Mupiderm (registered trademark)), a gel (for example, Finacea (registered trademark)), a spray (for example, Nasonex (registered trademark)), a patch (for example, Durogesic (registered trademark)), a lozenge (for example, Strepsils (registered trademark)), a solution for intravenous perfusion (for example, Salbutamol Merck (registered trademark)), for peridural, intra- or peri-articular, peripheral, regional or paravertebral administration (for example, Lidocaine Adrenaline Aguettant (registered trademark) 2% Injectable Solution), or intrathecal administration (for example, Bupiforan (registered trademark) 0.25% Injectable Solution), or infiltration, for example by local infiltration (for example, Bupivacaine Aguettant (registered trademark) 0.25% Injectable Solution). In these examples, the agonist can be added to or replace the active ingredients of the drugs mentioned. The compositions of each of these drugs can be found, for example, in the Vidal dictionary, edition 2007.

The pharmaceutically acceptable carrier may be, for example, any known pharmaceutically acceptable carrier used for administering the abovementioned agonists to a human or to an animal, according to the use of the medicine. For example, the carrier may be chosen from the group comprising povidone, colloidal silica, talc, microcrystalline cellulose, lactose monohydrate, gelatin, lecithin, starch, crospovidone, glycerol, paraffin, butylhydroxyanisole, petroleum jelly, lanolin and gum arabic.

The pharmaceutically acceptable medium may be any known medium which is suitable for the administration of an agonist, as defined in the present application, to a human or to an animal. This medium may, for example, be ethanol, sucrose, glycerol, propylene glycol, sodium saccharin, sodium acetate, acetic acid, water, a saline solution, liquid paraffin, butylhydroxyanisole, petroleum jelly, lanolin or gum arabic.

According to the invention, the administration is preferably chronic. The term “chronic administration” or “chronic treatment” is intended to mean, in the present application, an intake that is repeated or is maintained over at least several days, for example several weeks, for example several months, for example several years. For example, a chronic administration may consist of several intakes or administration of the drug in any one of its forms described above; or of a single or repeated intake of the drug in a long-acting form or a form with a duration of action maintained over time, for instance a drip or a patch. For example, a chronic administration can result in one, two, three or more intakes or administrations per day, for example for one day, two days, three days, etc., for example one week, two weeks, three weeks, etc., for example one month, two months, three months, etc., for example one year, two years, three years, etc.

According to the invention, the drug may comprise any dose of agonist that is pharmaceutically acceptable and effective in the implementation of the present invention. For example, the drug may comprise a dose enabling an administration, for example, in humans ranging from 0.01 to 120 mg/day, for example from 0.01 to 20 mg/day. This may, for example, comprise doses of from 10 to 20 mg/day for bambuterol, or from 0.02 to 0.2 mg/day for clenbuterol, or from 0.05 to 0.8 mg/day for fenoterol, or from 0.01 to 0.05 mg/day for formoterol, or from 0.2 to 10 mg/day for isoproterenol (or isoprenalin), or from 0.2 to 3 mg/day for pirbuterol, or from 0.05 to 72 mg/kg for ritodrin, for example from 0.05 to 120 mg/day, or from 0.1 to 36 mg/day for salbutamol, or from 0.025 to 0.1 mg/day for salmeterol, from 0.5 to 13.5 mg/day for terbutalin, 0.5 to 60 mg/day for metaproterenol, from 0.3 to 4 mg/day for levalbuterol, from 0.5 to 12 mg/day for reproterol, from 1 to 6 mg/day for tulobuterol, or from 0.5 to 10 mg/day for bitolterol. These are, of course, examples; the doses may be adjusted in particular according to the sensitivity of the patients to this treatment and the method of administration.

The present invention also relates to a method for treating pain, in particular neuropathic pain, for example neuropathic allodynia, this method comprising the administration, to a patient, of an agonist as defined above. The patient may be human or animal. Agonists that can be used and formulations are given as examples above.

The administration can be carried out by any means known to those skilled in the art, in particular by any known means for administering the abovementioned agonists. Examples of methods of administration are described below.

For example, the administration can be carried out by direct injection of the agonist, or by perfusion, by oral intake in the form in particular of a syrup, an oral solution, a tablet, an effervescent, or film-coated, or breakable, or coated, or gastro-resistant or dispersible or chewable tablet, a blister pack tablet, a gel capsule, a lozenge, a powder or granules for oral solution, through the use of a suspension or of a powder for inhalation, of a powder for inhalation in a gel capsule, or of a solution for inhalation by nebulizer, by taking a suppository, through the use of an eye lotion, through the use of a cream, an ointment or a gel, through the use of a spray, through the use of patches, by peridural, intra- or peri-articular, or regional,or peripheral, or intrathecal,or paravertebral or local administration or infiltration or perfusion. Examples of compositions that can be used for these various routes of administration are described above.

The administration can be defined in such a way as to enable a pharmaceutically acceptable and effective delivery for treating pain, in particular neuropathic pain. For example, the administration may comprise a dose, for example, in humans ranging from 0.01 to 120 mg/day, for example from 0.01 to 20 mg/day. This may, for example, comprise doses of from 10 to 20 mg/day for bambuterol, or from 0.02 to 0.2 mg/day for clenbuterol, or from 0.05 to 0.8 mg/day for fenoterol, or from 0.01 to 0.05 mg/day for formoterol, or from 0.2 to 10 mg/day for isoproterenol (or isoprenalin), or from 0.2 to 3 mg/day for pirbuterol, or from 0.05 to 72 mg/kg for ritodrin, for example from 0.05 to 120 mg/day, or from 0.1 to 36 mg/day for salbutamol, or from 0.025 to 0.1 mg/day for salmeterol, or from 0.5 to 13.5 mg/day for terbutalin. These are, of course, examples; the doses may be adjusted, in particular according to the sensitivity of the patients to this treatment.

The administration may be carried out as a single administration or as several administrations.

The administration of beta-adrenergic agonists according to the method of the invention may, for example, be a chronic administration.

The doses of beta-adrenergic agonists administered, for example chronically, may be between 0.01 and 20 mg/day, for example between 0.05 and 15 mg/day, for example between 0.05 and 10 mg/day, for example between 0.1 and 5 mg/day, or else, for example, between 0.15 and 350 microg/kg and per day, for example between 0.8 and 250 microg/kg and per day, for example between 0.8 and 170 microg/kg and per day, for example between 1.5 and 90 microg/kg and per day.

The action of the beta- or beta2-adrenergic agonists in the use in accordance with the present invention is more precise at the molecular level than that of antidepressants for treating pain, for example neuropathic allodynia, since these agonists can act directly on the receptor responsible for the therapeutic effect without affecting the other adrenergic receptors. This more targeted action definitely reduces the adverse side effects.

The present invention relates to the use of beta-adrenergic agonists for the treatment of neuropathic pain, for example neuropathic allodynia, in particular, and without being limited thereto, over a long period of time. Specifically, the experimental data obtained by the inventors from their animal model of neuropathic pain, in particular of neuropathic allodynia, which is extremely close to the clinical situation in humans, clearly show a considerable improvement in the pain symptoms after treatment with such molecules.

In addition, these molecules are already used in the human clinical field for treating other conditions, which facilitates and especially speeds up the first therapeutic trials on neuropathic pain. The therapeutic prospects are therefore immediate. Many beta- or beta2-adrenergic agonist molecules have already obtained a marketing authorization and are commonly used in the case of other pathological conditions, essentially asthma attacks or chronic obstructive pulmonary diseases, or for inhibiting uterine contractions. However, these molecules have never yet been tested against neuropathic pain, for example against neuropathic allodynia. The existence of these authorizations means that the clinical tests on side effects have already been carried out and should potentially promote/accelerate, at less expense, the progress to trials and to clinical use in the context of neuropathic pain.

Moreover, the efficacy of a chronic treatment is highly dependent on the patient accepting and following the chronic treatment. Psychologically, it may be easier to accept being treated with a molecule known to be prescribed against asthma than with a molecule for psychiatric purposes, known to be prescribed against depression.

Other advantages may become apparent to those skilled in the art on reading the examples which follow, which are obviously given by way of nonlimiting illustration, with reference to the attached figures.

### BRIEF DESCRIPTION OF THE FIGURES

FIG. 1 is a graph showing the effect of a saline solution (“Sal”) or of nortriptylin (“Nor”) on control mice (“Sham”) without cuff or neuropathic mice with cuff (“Cuff”) described in examples 1 and 2. Along the y-axis, the pressure exerted is expressed in grams (g) and along the x-axis, the time is expressed in days starting from the surgical procedure described in example 1.

FIG. 2 is a graph showing, in control (“Sham”) or neuropathic (“Cuff”) animals, the effect of a cotreatment with a saline solution (“Sal”) or a nortriptylin solution (“Nor”) and with the alpha2-adrenergic antagonist yohimbin or the beta-adrenergic antagonist propranolol, described in example 3. Along the y-axis, the pressure exerted is expressed in grams (g) and along the x-axis, the time is expressed in days starting from the surgical procedure described in example 1.

FIG. 3 comprises two graphs. The first shows, in neuropathic animals, the effect of a cotreatment with nortriptylin and with the beta1-adrenergic antagonist metoprolol, with the beta2-adrenergic antagonist ICI 118,551 or with the beta3-adrenergic antagonist SR59230A, described in examples 4, 5 and 6. Along the y-axis, the pressure exerted is expressed in grams (g). The second graph details the effect of a cotreatment with nortriptylin and with the beta2-adrenergic antagonist ICI 118,551, described in example 5. Along the y-axis, the pressure exerted is expressed in grams (g) and along the x-axis, the time is expressed in days starting from the surgical procedure described in example 1.

FIG. 4 is a graph showing, in control (“Sham”) or neuropathic (“Cuff”) animals, the effect of the first injection (“acute injection”) of clenbuterol described in example 7. Along the y-axis, the pressure exerted is expressed in grams (g) and along the x-axis, the time is expressed in minutes after the injection.

FIG. 5 is a graph showing, in control (“Sham”) or neuropathic (“Cuff”) animals, the effect of a treatment with clenbuterol (“Clen”) described in example 7. Along the y-axis, the pressure exerted is expressed in grams (g) and along the x-axis, the time is expressed in days starting from the surgical procedure described in example 1.

FIG. 6 is a graph showing, in control (“Sham”) or neuropathic (“Cuff”) animals, the effect of a cotreatment with clenbuterol and with the beta2-adrenergic antagonist ICI 118,551 described in example 8. Along the y-axis, the pressure exerted is expressed in grams (g) and along the x-axis, the time is expressed in days starting from the surgical procedure described in example 1.

FIG. 7 is a graph showing, in control (“Sham”) or neuropathic (“Cuff”) animals, the effect of the first injection (“acute injection”) of bambuterol described in example 9. Along the y-axis, the pressure exerted is expressed in grams (g) and along the x-axis, the time is expressed in minutes after the injection.

FIG. 8 is a graph showing, in control (“Sham”) or neuropathic (“Cuff”) animals, the effect of a treatment with bambuterol described in example 9. Along the y-axis, the pressure exerted is expressed in grams (g) and along the x-axis, the time is expressed in days starting from the surgical procedure described in example 1.

FIG. 9 is a graph showing, in control (“Sham”) or neuropathic (“Cuff”) animals, the effect of a treatment with fenoterol described in example 10. Along the y-axis, the pressure exerted is expressed in grams (g) and along the x-axis, the time is expressed in days starting from the surgical procedure described in example 1.

FIG. 10 is a graph showing, in control (“Sham”) or neuropathic (“Cuff”) animals, the effect of a treatment with formoterol at the dose of 0.5 mg/kg described in example 11. Along the y-axis, the pressure exerted is expressed in grams (g) and along the x-axis, the time is expressed in days starting from the surgical procedure described in example 1.

FIG. 11 is a graph showing, in control (“Sham”) or neuropathic (“Cuff”) animals, the effect of the first injection (“acute injection”) of formoterol at the dose of 0.05 mg/kg described in example 11. Along the y-axis, the pressure exerted is expressed in grams (g) and along the x-axis, the time is expressed in minutes after the injection.

FIG. 12 is a graph showing, in control (“Sham”) or neuropathic (“Cuff”) animals, the effect of treatments with formoterol at various doses (0.05 mg/kg; 0.005 mg/kg; 0.0005 mg/kg) described in example 11. Along the y-axis, the pressure exerted is expressed in grams (g) and along the x-axis, the time is expressed in days starting from the surgical procedure described in example 1.

FIG. 13 is a graph showing, in control (“Sham”) or neuropathic (“Cuff”) animals, the effect of a treatment with salbutamol described in example 12. Along the y-axis, the pressure exerted is expressed in grams (g) and along the x-axis, the time is expressed in days starting from the surgical procedure described in example 1.

FIG. 14 is a graph showing, in control (“Sham”) or neuropathic (“Cuff”) animals, the effect of a treatment with salbutamol by inhalation described in example 12. Along the y-axis, the pressure exerted is expressed in grams (g). Along the x-axis, the time is expressed in days before surgery as described in example 1, before the beginning of the treatment and at the end of the treatment, as described in example 12.

FIG. 15 is a graph showing, in control (“Sham”) or neuropathic (“Cuff”) animals, the effect of a treatment with salmeterol described in example 13. Along the y-axis, the pressure exerted is expressed in grams (g) and along the x-axis, the time is expressed in days starting from the surgical procedure described in example 1.

FIG. 16 is a graph showing, in control (“Sham”) or neuropathic (“Cuff”) animals, the effect of a treatment with salmeterol by inhalation described in example 13. Along the y-axis, the pressure exerted is expressed in grams (g). Along the x-axis, the time is expressed in days before surgery as described in example 1, before the beginning of the treatment and at the end of the treatment, as described in example 12.

FIG. 17 is a graph showing, in control (“Sham”) or neuropathic (“Cuff”) animals, the effect of a treatment with terbutalin described in example 14. Along the y-axis, the pressure exerted is expressed in grams (g) and along the x-axis, the time is expressed in days starting from the surgical procedure described in example 1.

FIG. 18 is a graph showing, in control (“Sham”) or neuropathic (“Cuff”) animals, the effect of a treatment with terbutalin (0.5 mg/kg) or with saline described in example 14. The left-hand graph shows the results of the left foot (without surgery). The right-hand graph shows the results of the right foot (with surgery). Along the y-axis, the pressure exerted is expressed in grams (g) and along the x-axis, the time is expressed in days starting from the surgical procedure described in example 1.

FIG. 19 is a graph showing, in control (“Sham”) or neuropathic (“Cuff”) animals, the effect of a cotreatment with terbutalin at the dose of 0.5 mg/kg and with the beta2-adrenergic antagonist ICI 118,551 described in example 14. Along the y-axis, the pressure exerted is expressed in grams (g).

FIG. 20 is a graph showing, in control (“Sham”) or neuropathic (“Cuff”) animals, the effect of treatments with terbutalin at various doses (0.25 mg/kg; 0.125 mg/kg; 0.05 mg/kg; 0 mg/kg) described in example 14. Along the y-axis, the pressure exerted is expressed in grams (g) and along the x-axis, the time is expressed in days starting from the surgical procedure described in example 1.

FIG. 21 is a graph recapitulating, in control (“Sham”) or neuropathic (“Cuff”) animals, the compared effect of a chronic treatment with the control solution (saline, “dose 0”), with terbutalin at various doses and with nortriptylin described in example 14. The top graph gives the results of the left foot (without surgery). The bottom graph gives the results of the right foot (with surgery). Along the y-axis, the recovery of the animals is indicated as percentage of their sensitivity before surgery.

FIG. 22 is a graph showing, in control (“Sham”) or neuropathic (“Cuff”) animals, the effect of a treatment with terbutalin (Ter) using a gel described in example 14. The control gel does not contain terbutalin. Along the y-axis, the pressure exerted is expressed in grams (g) and along the x-axis, the time is expressed in days starting from the surgical procedure described in example 1.

FIG. 23 is a graph showing, in control (“Sham”) or neuropathic (“Cuff”) animals, the effect of the fitting of an implant of terbutalin (Ter) described in example 14. The control implant does not contain terbutalin. Along the y-axis, the pressure exerted is expressed in grams (g) and along the x-axis, the time is expressed in days starting from the surgical procedure described in example 1.

FIG. 24 is a graph showing, in control (“Sham”) or neuropathic (“Cuff”) animals, the effect of the first injection (“acute injection”) of fenoterol, of salbutamol, of salmeterol and of terbutalin described in example 15. Along the y-axis, the pressure exerted is expressed in grams (g) and along the x-axis, the time is expressed in minutes after the injection.

FIG. 25 is a graph recapitulating, in neuropathic (“Cuff”) animals, the compared effect of a chronic treatment with the control solution (saline), with nortriptylin, with bambuterol, with clenbuterol, with fenoterol, with formoterol, with salbutamol, with salmeterol, with terbutalin, with isoprenalin, with ritodrin, with metaproterenol and with procaterol described in example 16. Along the y-axis, the recovery of the animals is indicated as percentage of their sensitivity before surgery.

FIG. 26 is a graph recapitulating, in control (“Sham”) animals, the compared effect of a chronic treatment with the control solution (saline), with nortriptylin, with bambuterol, with clenbuterol, with fenoterol, with formoterol, with salbutamol, with salmeterol, with terbutalin, with isoprenalin, with ritodrin, with metaproterenol and with procaterol described in example 17. Along the y-axis, the nociceptive threshold of the animals is indicated as percentage of their sensitivity before surgery.

FIG. 27 is a graph showing, in control (“Sham”) or neuropathic (“Cuff”) animals, the effect of a cotreatment with bambuterol or with fenoterol or with formoterol or with salmeterol, and with the beta2-adrenergic antagonist ICI 118,551 described in example 18. Along the y-axis, the pressure exerted is expressed in grams (g).

FIG. 28 is a graph showing, in control (“Sham”) or neuropathic (“Cuff”) animals, the effect of a cotreatment with salbutamol and with the beta2-adrenergic antagonist ICI 118,551 injected either intraperitoneally or intrathecally, described in example 19. Along the y-axis, the pressure exerted is expressed in grams (g).

FIG. 29 is a graph showing, in control (“Sham”) or neuropathic (“Cuff”) animals, the effect of a treatment with isoprenalin (or isoproterenol) described in example 20. Along the y-axis, the pressure exerted is expressed in grams (g) and along the x-axis, the time is expressed in days starting from the surgical procedure described in example 1.

FIG. 30 is a graph showing, in control (“Sham”) or neuropathic (“Cuff”) animals, the effect of a cotreatment with isoprenalin and with the beta2-adrenergic antagonist ICI 118,551 described in example 20. Along the y-axis, the pressure exerted is expressed in grams (g).

FIG. 31 is a graph showing, in control (“Sham”) or neuropathic (“Cuff”) animals, the effect of a treatment with ritodrin described in example 20. Along the y-axis, the pressure exerted is expressed in grams (g) and along the x-axis, the time is expressed in days starting from the surgical procedure described in example 1.

FIG. 32 is a graph showing, in control (“Sham”) or neuropathic (“Cuff”) animals, the effect of a cotreatment with ritodrin and with the beta2-adrenergic antagonist ICI 118,551 described in example 20. Along the y-axis, the pressure exerted is expressed in grams (g).

FIG. 33 is a graph showing, in control (“Sham”) or neuropathic (“Cuff”) animals, the effect of a treatment with metaproterenol described in example 20. Along the y-axis, the pressure exerted is expressed in grams (g) and along the x-axis, the time is expressed in days starting from the surgical procedure described in example 1.

FIG. 34 is a graph showing, in control (“Sham”) or neuropathic (“Cuff”) animals, the effect of a treatment with metaproterenol using a gel described in example 20. Along the y-axis, the pressure exerted is expressed in grams (g) and along the x-axis, the time is expressed in days starting from the surgical procedure described in example 1.

FIG. 35 is a graph showing, in control (“Sham”) or neuropathic (“Cuff”) animals, the effect of a treatment with procaterol described in example 20. Along the y-axis, the pressure exerted is expressed in grams (g) and along the x-axis, the time is expressed in days starting from the surgical procedure described in example 1.

### EXAMPLES

In the examples which follow, the experimental protocols and results illustrate the scientific data of the inventors which lead to proposing beta-adrenergic agonists for treating neuropathic pain, in particular neuropathic allodynia. The inventors demonstrate experimentally, in particular:
1. that repeated blocking of alpha2-adrenergic receptors (for example with yohimbin) is ineffective in blocking the analgesic effect of antidepressants: this effect does not therefore involve alpha2-adrenoreceptors;
2. that repeated blocking of beta-adrenergic receptors (for example with propranolol) blocks the therapeutic effect of antidepressants: beta-adrenoreceptors are therefore responsible for the therapeutic effect of antidepressants on neuropathic pain, for example on neuropathic allodynia. This effect is specific since propranolol does not affect the response of the control animals, treated or not treated, for example, with nortriptylin.

The inventors also test molecules specific for the 3 subtypes of beta-adrenoreceptors: metoprolol against the beta1-adrenoreceptors; ICI 118,551 against the beta2-adrenoreceptors and SR59230A against the beta3-adrenoreceptors, and show that only the beta2-adrenoreceptor antagonist blocks the effect of antidepressants. This demonstrates that the therapeutic effect of antidepressants against neuropathic pain, for example against neuropathic allodynia, requires beta2-adrenoreceptors. This effect is specific since ICI 118,551 does not affect the response of the control animals, treated or not treated with nortriptylin.

The inventors then demonstrate that a chronic treatment with various beta2-adrenoreceptor-specific agonists causes the neuropathic pain, for example the neuropathic allodynia, to completely disappear, and can therefore be used as a substitute for the use of the antidepressants. This demonstration was made, for example, with clenbuterol, fenoterol, formoterol, salbutamol, salmeterol, terbutalin, ritodrin, metaproterenol and procaterol. The same demonstration was made with, for example, bambuterol and isoprenalin, which are general beta-adrenergic agonists. Beta2-adrenergic agonists, and more generally beta-adrenergic agonists, can therefore enable neuropathic pain, in particular neuropathic allodynia, to be treated.

The inventors demonstrate that this effect of beta2-adrenoreceptor agonists on neuropathic pain, for example on neuropathic allodynia, is not observed acutely after the first injection of the agonist under consideration. This was demonstrated, for example, with clenbuterol, bambuterol, fenoterol, formoterol, salbutamol, salmeterol and terbutalin. A chronic treatment is therefore necessary.

The inventors show that the chronic treatment with various beta2-adrenoreceptor-specific agonists does not aspecifically affect the sensitivity of the control animals. This demonstration was made, for example, with clenbuterol, fenoterol, formoterol, salbutamol, salmeterol, terbutalin, ritodrin, metaproterenol and procaterol. The same demonstration was made, for example, with bambuterol and isoprenalin, which are general beta-adrenergic agonists. At therapeutic doses, the beta2-adrenergic agonists, and more generally the beta-adrenergic agonists, do not therefore in the long term have any aspecific effect on nociceptive sensitivity.

The inventors demonstrate that the beta2-adrenoreceptor antagonist ICI 118,551 blocks the therapeutic effect of a chronic treatment with beta-adrenergic receptor agonists on neuropathic pain, for example on neuropathic allodynia. This demonstration was made, for example, with bambuterol, isoprenalin, clenbuterol, fenoterol, formoterol, salbutamol, salmeterol, terbutalin, ritodrin and procaterol. The effect of the treatment with these agonists on neuropathic pain, for example on neuropathic allodynia, therefore clearly involves their action on beta2-adrenoreceptors.

The inventors demonstrate that the beta2-adrenoreceptor antagonist ICI 118,551 delivered intrathecally blocks the therapeutic effect of a chronic treatment with a beta-adrenergic receptor agonist. This demonstration was made, for example, with salbutamol. These results show that the spinal cord and/or the dorsal root ganglia are involved in the therapeutic effect of beta2-adrenergic agonists.

The inventors demonstrate that the administration by inhalation of beta2-adrenoreceptor agonists, for instance salbutamol or salmeterol, relieves neuropathic allodynia.

The inventors demonstrate that the transcutaneous administration, in the form of a gel, of beta2-adrenoreceptor agonists, for instance terbutalin or metaproterenol, relieves neuropathic allodynia.

The inventors demonstrate that the continuous delivery of beta2-adrenoreceptor agonists, such as with, for example, terbutalin implants, relieves neuropathic allodynia.

The details of the operating protocols and results obtained are given in the examples below.

### Example 1

### Murine Model of Neuropathic Pain

The experiments were carried out on adult male mice. The pharmacological experiments were carried out using C57BL/6J mice (3-to-6 weeks old on their arrival, Charles River, L'Arbresle, France).

All the experiments began with mice that were 6-to-9 weeks old. For the gel application experiments, the mice were housed individually. For all the other experiments, the mice were combined at four to five per cage and kept under alternating 12-hour day/night (light at 6 am) with food and water ad libitum. The procedures were carried out according to guideline 86/6609/EEC.

The model is obtained by placing a polyethylene cuff around the main branch of the right sciatic nerve of each mouse. Before any surgical procedure, the mice were assigned to the various experimental groups in such a way that the baseline mechanical nociception threshold and the weight are equivalent between the various groups. The surgical procedure was carried out under ketamin-xylazin anesthesia (ketamin: 17 mg/ml, xylazin 2.5 mg/ml, intraperitoneal injection in a proportion of 4 ml/kg) (limited liability company Centravet, Taden, France). The common branch of the sciatic nerve was exposed and a section 2 mm long of a hollow PE-20 polyethylene tube (“Harvard Apparatus”, Les Ulis, France) was placed around said branch (“Cuff” group). This protocol was published previously (Benbouzid, M. et al. Biological Psychiatry 63:633-636 (2008) (10) and Benbouzid, M. et al. European Journal of Pain 12:591-599 (2008) (11)). Control mice were operated on according to the same procedure, but without placing the cuff (“Sham” group). The experimental groups are made up of at least 4 mice for each control (“Sham”) group and at least 5 mice for each neuropathic (“Cuff”) group.

The placing of the polyethylene cuff (“Cuff” group) around the main branch of the sciatic nerve leads to mechanical allodynia, i.e. a response to a stimulation that is not normally painful, compared with the control animals (“control” or “Sham” group). This means that the threshold for pressure leading to a foot withdrawal response by the mouse greatly decreases, as indicated by the graph of FIG. 1.

For the tests, in the examples which follow, the pressure is exerted on the foot of the mice by means of von Frey filaments (Bioseb, Chaville, France) (Benbouzid, M. et al. Biological Psychiatry 63:633-636 (2008) (10) and Benbouzid, M. et al. European Journal of Pain 12:591-599 (2008) (11)). The mice are placed in transparent Plexiglas (registered trademark) boxes (7 cm×9 cm×7 cm) placed on a raised grid. Before the test, the mice are allowed to become accustomed to this device for 15 minutes. During the test, the von Frey filaments are applied to the plantar surface of each back foot according to ascending forces (from 0.16 g up to a foot withdrawal response, or up to a maximum of 10 g). Each filament is tested 5 times per foot and the threshold is defined by the presence of 3 or more withdrawals observed over the 5 tests. Before the surgery as described above, the animals were pretested in order to establish their baseline nociceptive response. During the chronic treatment experiments and those of repeated injection of antagonists, the tests are carried out in the mornings before the first injection of the day. During the acute agonist injection experiments, the tests are carried out after the first injection according to the time course as presented on the graphs.

The surgery for installing the cuff corresponds to day 0. The mechanical allodynia obtained on these mice lasts more than 2 months as previously published (Benbouzid, M. et al. Biological Psychiatry 63:633-636 (2008) (10) and Benbouzid, M. et al. European Journal of Pain 12:591-599 (2008) (11)).

This neuropathic mouse model is sensitive to chronic treatment with a tricyclic antidepressant (nortriptylin or amitriptylin) as previously published (Benbouzid, M. et al. Biological Psychiatry 63:633-636 (2008) (10)).

For all the treatments carried out by injection, said injections were given intraperitoneally in a volume of 50 microL per 10 mg of body weight.

### Example 2

### Therapeutic Effect of a Tricyclic Antidepressant

On mouse models obtained in the manner described in example 1, when allodynia was correctly in place, on day 15 after the procedure for installing the cuff, a therapeutic treatment was begun with the antidepressant nortriptylin or “Nor”, in a proportion of 5 mg/kg (nortriptylin hydrochloride; available from suppliers such as, for example, Sigma-Aldrich, catalog number N7261; diluted in a saline solution containing 0.9% of NaCl), injection twice a day (morning and evening). This treatment reproduces what is observed clinically: the treatment is ineffective initially, but subsequently causes the allodynia to completely disappear.

A “placebo” group of mice was subjected to an injection of saline solution only (0.9% NaCl solution), without antidepressant (on the graph “Sal” for: injection of 0.9% saline solution). This “Sal” group remains allodynic throughout the experiment.

The results of this example are shown in the attached FIG. 1.

The nociceptive sensitivity of the mice of the control (or “Sham” group) is not affected by the treatment with nortriptylin: their nociceptive threshold remains stable throughout the experiment.

### Example 3

### Absence of Effect of Blocking Alpha2-Adrenoreceptors on the Action of the Antidepressant and Presence of an Effect of Blocking Beta-Adrenoreceptors on the Action of the Antidepressant

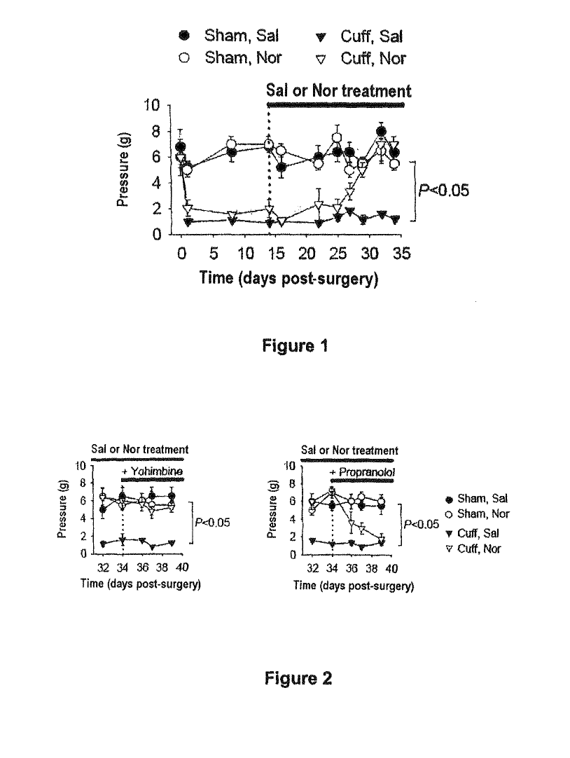

The left-hand graph of FIG. 2 shows that, in neuropathic (“Cuff”) animals which were treated for 3 weeks with nortriptylin (5 mg/kg, twice a day) as described in example 2 (and therefore experiencing relief of their allodynia), a cotreatment with nortriptylin and the alpha2-adrenoreceptor antagonist (blocker) (yohimbin, 2 mg/kg, intraperitoneal) (yohimbin hydrochloride; available from suppliers such as, for example, Sigma-Aldrich, catalog number Y3125; diluted in a saline solution containing 0.9% NaCl), has no influence on the therapeutic benefit. The therapeutic effect of nortriptylin does not therefore involve recruitment of alpha2-adrenoreceptors.

Blocking the beta-adrenoreceptors suppresses the therapeutic effect of the antidepressant.

The right-hand graph of FIG. 2 shows that, in neuropathic animals treated for 3 weeks with nortriptylin (5 mg/kg, twice a day), a cotreatment with nortriptylin as described in example 2 and the beta-adrenoreceptor antagonist (propranolol, 5 mg/kg, intraperitoneal) ((+/−)-propranolol hydrochloride; available from suppliers such as, for example, Sigma-Aldrich, catalog number P0884; diluted in a saline solution containing 0.9% NaCl) causes the allodynia to reappear in a few days. The therapeutic effect of the antidepressant therefore involves beta-adrenoreceptors.

The nociceptive sensitivity of the mice of the control (or “Sham”) group is not affected by the yohimbin or propranolol injections: their nociceptive threshold remains stable throughout the experiment.

The same results are obtained using sotatol, 2 mg/kg, ((+/−) sotatol hydrochloride; available from suppliers such as, for example, Sigma-Aldrich, catalog number S0278; diluted in a saline solution containing 0.9% NaCl), in place of the propranolol, 5 mg/kg.

### Example 4

### Absence of Effect of Blocking Beta1-Adrenoreceptors on the Action of the Antidepressant

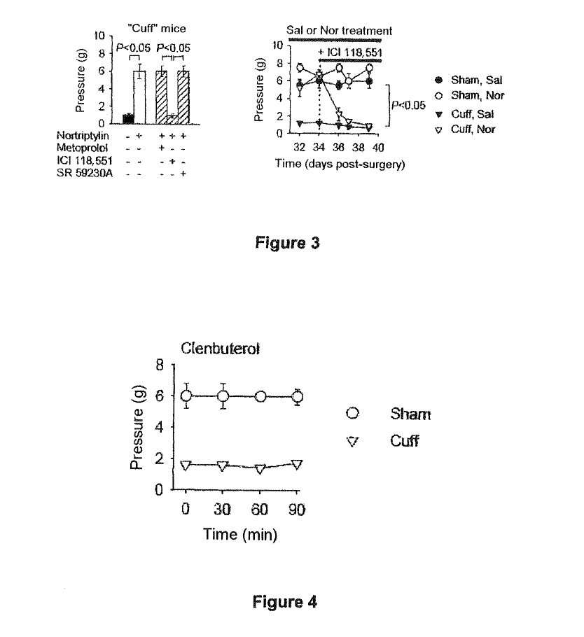

The left-hand graph of FIG. 3 shows that, in neuropathic animals treated for 3 weeks with nortriptylin as described in example 2, a cotreatment, for 6 days, with nortriptylin and the beta1-adrenoreceptor antagonist (metoprolol, 2 mg/kg, intraperitoneal) ((+/−)-metoprolol (+)-tartrate; available from suppliers such as, for example, Sigma-Aldrich, catalog number M5391; diluted in a saline solution containing 0.9% NaCl) has no influence on the therapeutic benefit.

The same results are obtained using atenolol, 5 mg/kg (atenolol; available from suppliers such as, for example, Sigma-Aldrich, catalog number A7655; diluted in a saline solution containing 0.9% NaCl), in place of the metoprolol, 2 mg/kg.

### Example 5

### Presence of an Effect of Blocking Beta2-Adrenoreceptors on the Action of the Antidepressant

The left-hand graph of FIG. 3 shows that, in neuropathic animals treated for 3 weeks with nortriptylin as described in example 2, a cotreatment, for 6 days, with nortriptylin and the beta2-adrenoreceptor antagonist (ICI 118,551, 2 mg/kg, intraperitoneal) (ICI 118,551 hydrochloride; available from suppliers such as, for example, Sigma-Aldrich, catalog number I127; diluted in a saline solution containing 0.9% NaCl) leads to reappearance of the neuropathic allodynia.

Blocking the beta2-adrenoreceptors suppresses the therapeutic effect of the antidepressant.

The right-hand graph of FIG. 3 details the time course of the reappearance of neuropathic pain in the mice bearing the cuff (“Cuff”) when ICI 118,551 is coadministered with nortriptylin (“Nor”), as described above. It shows that the allodynia reappears within 48 hours following the beginning of the cotreatment.

The nociceptive sensitivity of the mice of the control (or “Sham”) group is not affected by the ICI 118,551 injections.

### Example 6

### Absence of Effect of Blocking Beta3-Adrenoreceptors on the Action of the Antidepressant

The left-hand graph of FIG. 3 shows that, in neuropathic animals treated for 3 weeks with nortriptylin, a cotreatment, for 6 days, with nortriptylin and the beta3-adrenoreceptor antagonist (SR59230A, 2.5 mg/kg, intraperitoneal) (SR59230A; available from suppliers such as, for example, Sigma-Aldrich, catalog number S8688; diluted in a saline solution containing 0.9% NaCl) has no influence on the therapeutic benefit.

### Example 7

### THERAPY: The Beta2-Adrenoreceptor Agonist Clenbuterol Relieves Neuropathic Allodynia

The graph of FIG. 4 shows that the first injection of the beta2-adrenergic agonist clenbuterol (0.3 mg/kg, intraperitoneal) (clenbuterol hydrochloride; available from suppliers such as, for example, Sigma-Aldrich, catalog number C5423; diluted in a saline solution containing 0.9% NaCl) has no effect on the mechanical allodynia of the neuropathic (“Cuff”) mice for 2 weeks.

The mice of the control (or “Sham”) group are also unaffected by the first administration of this agonist.

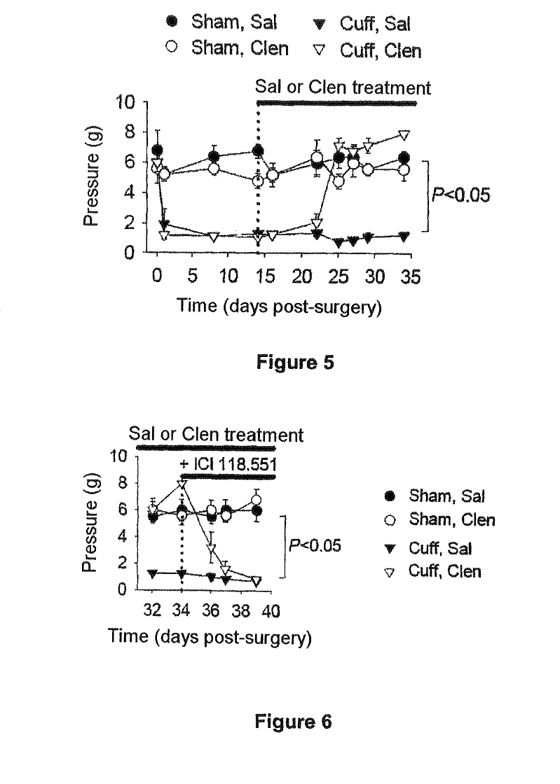

The graph of FIG. 5 shows that a treatment with the beta2-adrenergic agonist clenbuterol (“Clen”, 0.3 mg/kg, intraperitoneal, twice a day (morning and evening)) (clenbuterol hydrochloride; available from suppliers such as, for example, Sigma-Aldrich, catalog number C5423; diluted in a saline solution containing 0.9% NaCl) completely suppresses the mechanical allodynia in the neuropathic (“Cuff”) mice after chronic treatment.

The nociceptive sensitivity of the mice of the control (or “Sham”) group is not affected by the clenbuterol injections.

These data show for the first time that the administration of clenbuterol can relieve neuropathic pain, in particular neuropathic allodynia.

### Example 8

### Effect of Blocking Beta2-Adrenoreceptors on the Action of Clenbuterol

The graph of FIG. 6 shows that, after 3 weeks of treatment with clenbuterol as described in example 7, if clenbuterol and the beta2-adrenoreceptor antagonist ICI 118,551 (“ICI”, 2 mg/kg, intraperitoneal, as described in example 5) are coadministered, this causes the allodynia to reappear in the neuropathic (“Cuff”) animals in a few days. The therapeutic effect of clenbuterol therefore clearly involves its action on beta2-adrenoreceptors.

The nociceptive sensitivity of the mice of the control (or “Sham”) group is not affected by the ICI 118,551 injections.

### Example 9

### THERAPY: The Beta-Adrenoreceptor Agonist Bambuterol Relieves Neuropathic Allodynia

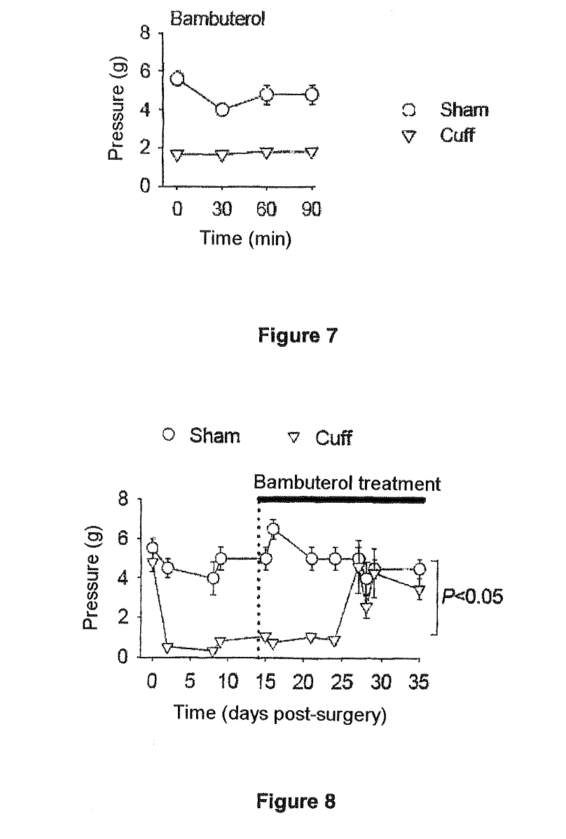

The graph of FIG. 7 shows that the first injection of the beta2-adrenergic agonist bambuterol (0.5 mg/kg, intraperitoneal) (bambuterol hydrochloride; available from suppliers such as, for example, Sigma-Aldrich, catalog number B8684; diluted in a saline solution containing 0.9% NaCl) has no anti-allodynia effect in neuropathic (“Cuff”) mice for 2 weeks.

The mice of the control (or “Sham”) group are also unaffected by the first administration of this agonist.

The graph of FIG. 8 shows that a treatment with the beta-adrenergic agonist bambuterol (0.5 mg/kg, twice a day (morning and evening)) (bambuterol hydrochloride; available from suppliers such as, for example, Sigma-Aldrich, catalog number B8684; diluted in a saline solution containing 0.9% NaCl) completely suppresses the mechanical allodynia in the neuropathic (“Cuff”) mice after chronic treatment.

The nociceptive sensitivity of the mice of the control (“Sham”) group is not affected by the bambuterol injections.

These data show for the first time that the administration of bambuterol can relieve neuropathic pain, in particular neuropathic allodynia.

### Example 10

### THERAPY: The Beta2-Adrenoreceptor Agonist Fenoterol Relieves Neuropathic Allodynia

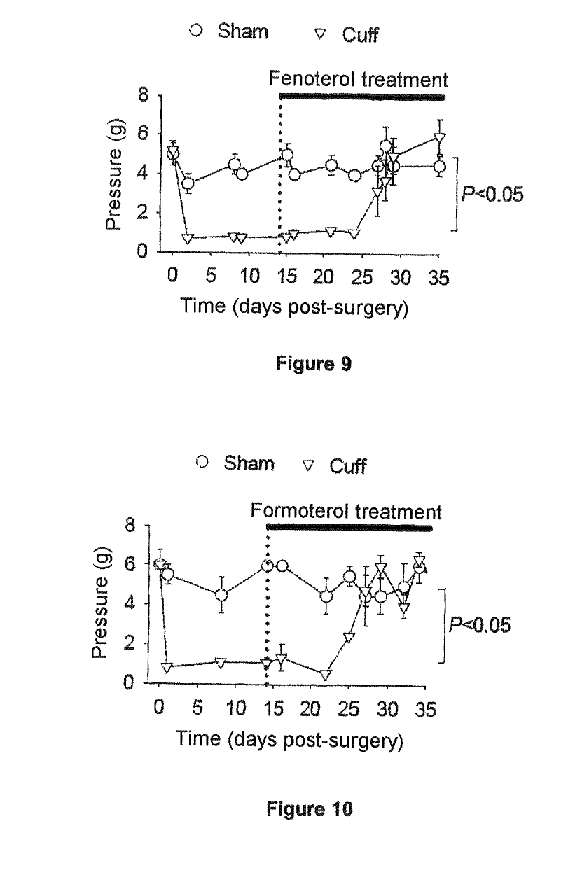

The graph of FIG. 9 shows that a treatment with the beta2-adrenergic agonist fenoterol (0.7 mg/kg, twice a day (morning and evening)) (fenoterol hydrobromide; available from suppliers such as, for example, Sigma-Aldrich, catalog number F1016; diluted in a saline solution containing 0.9% NaCl) completely suppresses the mechanical allodynia in the neuropathic (“Cuff”) mice after chronic treatment.

The nociceptive sensitivity of the mice of the control (or “Sham”) group is not affected by the fenoterol injections.

These data show for the first time that the administration of fenoterol can relieve neuropathic pain, in particular neuropathic allodynia.

### Example 11

### THERAPY: The Beta2-Adrenoreceptor Agonist Formoterol Relieves Neuropathic Allodynia

The graph of FIG. 10 shows that a treatment with the beta2-adrenergic agonist formoterol (0.5 mg/kg, injection twice a day (morning and evening)) (formoterol fumarate dihydrate; available from suppliers such as, for example, Sigma-Aldrich, catalog number F9552; diluted in a saline solution containing 0.9% NaCl) completely suppresses the mechanical allodynia in the neuropathic (“Cuff”) mice after chronic treatment.

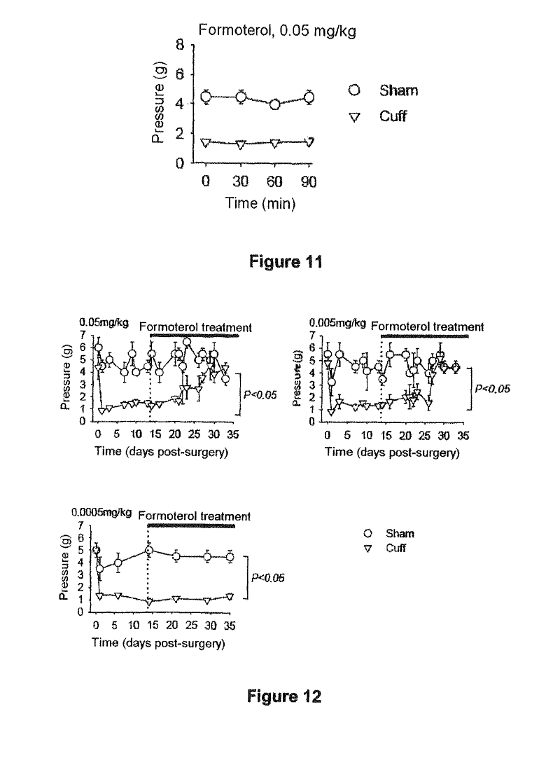

The graph of FIG. 11 shows that an acute injection of the beta2-adrenergic agonist formoterol (0.05 mg/kg, intraperitoneal) has no anti-allodynia effect in neuropathic (“Cuff”) mice for 2 weeks.

The mice of the control (or “Sham”) group are also unaffected by the first administration of this agonist at this dose.

The graphs of FIG. 12 show that treatments with the beta2-adrenergic agonist formoterol at the doses of 0.05 mg/kg or 0.005 mg/kg (injection twice a day (morning and evening)) completely suppress the mechanical allodynia of the right foot (neuropathic foot) after chronic treatment. The experimental groups are made up of 4 mice for all the control (“Sham”) groups, 5 mice for the neuropathic (“Cuff”) groups receiving the doses of 0.05 mg/kg or 0.005 mg/kg, and 4 mice for the neuropathic (“Cuff”) group receiving the dose of 0.0005 mg/kg.

The nociceptive sensitivity of the mice of the control (or “Sham”) group is not affected by the formoterol injections.

These data show for the first time that the administration of formoterol can relieve neuropathic pain, in particular neuropathic allodynia.

### Example 12

### THERAPY: The Beta2-Adrenoreceptor Agonist Salbutamol Relieves Neuropathic Allodynia

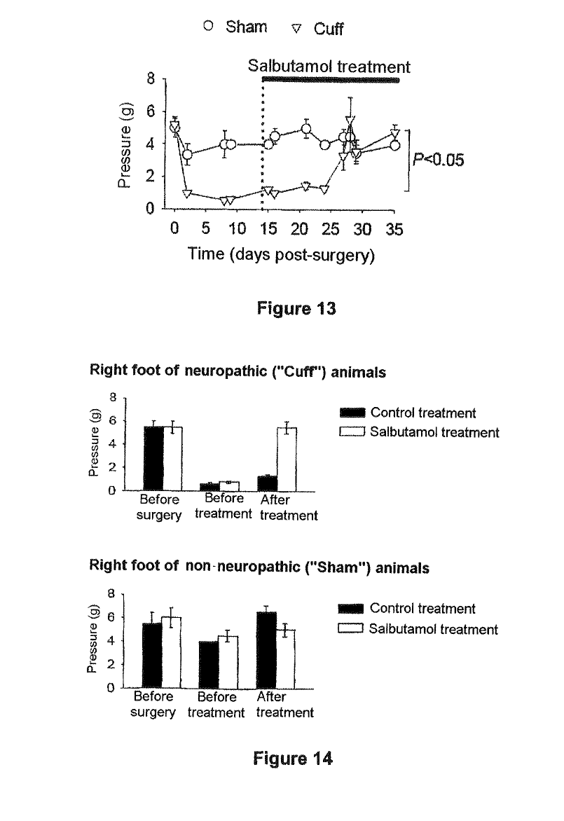

The graph of FIG. 13 shows that a treatment with the beta2-adrenergic agonist salbutamol (2 mg/kg, injection twice a day (morning and evening)) (salbutamol hemisulfate; available from suppliers such as, for example, Sigma-Aldrich, catalog number S5013; diluted in a saline solution containing 0.9% NaCl) relieves the mechanical allodynia in the neuropathic (“Cuff”) mice after chronic treatment.

The nociceptive sensitivity of the mice of the control (or “Sham”) group is not affected by the salbutamol injections.

These data show for the first time that the administration of salbutamol can relieve neuropathic pain, in particular neuropathic allodynia.

The graphs of FIG. 14 show that a treatment with the beta2-adrenergic agonist salbutamol by inhalation (twice a day (morning and evening)) (salbutamol sulfate in an inhaler; available from suppliers such as, for example, GlaxoSmithKline under the name Ventolin®) relieves the mechanical allodynia in the neuropathic (“Cuff”) mice after chronic treatment. In order to carry out this treatment, the animals were placed individually inside inhalation chambers (available from suppliers such as, for example, GlaxoSmithKline, Babyhaler® inhalation chamber reference 7072184), the main air outlets of which were blocked. The pressurized Ventolin® canister is connected to the chamber and pressing once makes it possible to deliver 100 μg of salbutamol per administration session (i.e. 200 μg per day) into the inhalation chamber when the muzzle of the mouse is close to the inhaler. The animal is then left in the chamber for 5 minutes before being removed and put back in its cage. The treatment began 2 weeks after the surgery for placing the cuffs around the sciatic nerve. After 2 weeks of treatment with the inhaler being pressed once per administration session, the treatment was changed, for 9 days, to the inhaler being pressed 3 times per administration session. The graph of figure 14 corresponds to the results before surgery, before the beginning of the treatment and on the last day of treatment. The control animals for the treatment (nontreated animals) follow the same procedure but without delivery of salbutamol.

These data show for the first time that an administration of salbutamol by inhalation can relieve neuropathic pain, in particular neuropathic allodynia.

### Example 13

### THERAPY: The Beta2-Adrenoreceptor Agonist Salmeterol Relieves Neuropathic Allodynia

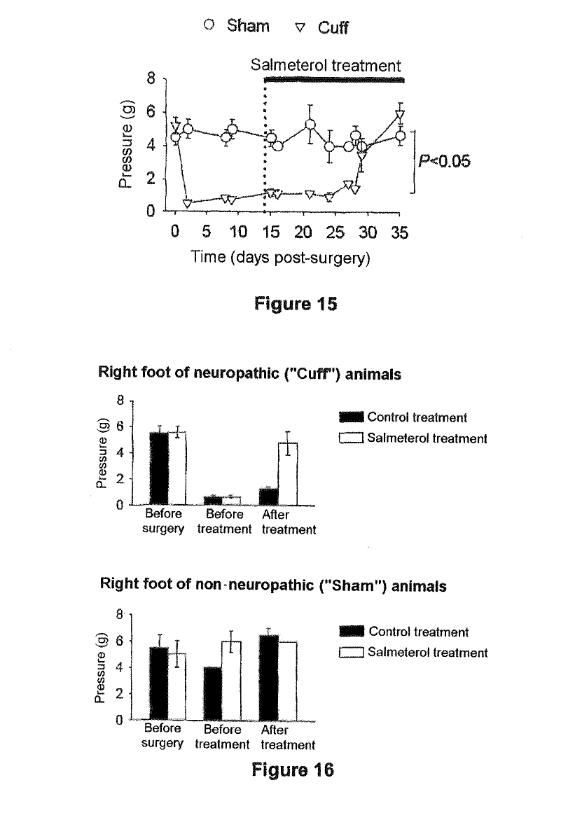

The graph of FIG. 15 shows that a treatment with the beta2-adrenergic agonist salmeterol (1 mg/kg, injection twice a day (morning and evening)) (salmeterol xinafoate; available from suppliers such as, for example, Sigma-Aldrich, catalog number S5068; diluted in a saline solution containing 0.9% NaCl) completely suppresses the mechanical allodynia after chronic treatment.

The nociceptive sensitivity of the mice of the control (or “Sham”) group is not affected by the salmeterol injections.

These data show for the first time that the administration of salmeterol can relieve neuropathic pain, in particular neuropathic allodynia.

The graphs of FIG. 16 show that a treatment with the beta2-adrenergic agonist salmeterol by inhalation (twice a day (morning and evening)) (salmeterol xinafoate in an inhaler; available from suppliers such as, for example, GlaxoSmithKline under the name Serevent®) relieves the mechanical allodynia in the neuropathic (“Cuff”) mice after chronic treatment. In order to carry out this treatment, the animals were placed individually inside inhalation chambers (available from suppliers such as, for example, GlaxoSmithKline, Babyhaler® inhalation chamber reference 7072184), the main air outlets of which were blocked. The pressurized Serevent® canister is connected to the chamber and pressing twice makes it possible to deliver 50 μg of salmeterol per administration session (i.e. 100 μg per day) into the inhalation chamber when the muzzle of the mouse is close to the inhaler. The animal is then left in the chamber for 5 minutes before being removed and put back in its cage. The treatment began 2 weeks after the surgery for placing the cuffs around the sciatic nerve. After 2 weeks of treatment with the inhaler being pressed twice per administration session, the treatment was changed, for 9 days, to the inhaler being pressed four times per administration session. The graph of FIG. 14 corresponds to the results before surgery, before the beginning of the treatment and on the last day of treatment. The control animals for the treatment (nontreated animals) follow the same procedure but without delivery of salmeterol.

These data show for the first time that administration of salmeterol by inhalation can relieve neuropathic pain, in particular neuropathic allodynia.

### Example 14

### THERAPY: The Beta2-Adrenoreceptor Agonist Terbutalin Relieves Neuropathic Allodynia

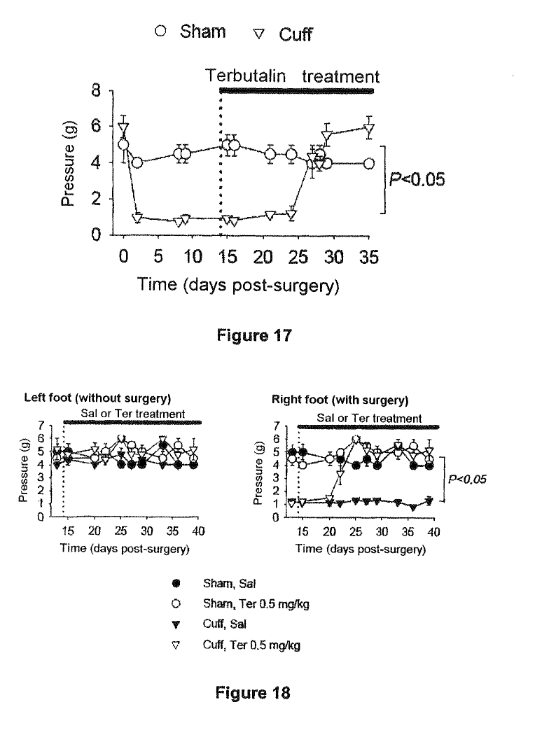

The graph of FIG. 17 shows that a treatment with the beta2-adrenergic agonist terbutalin (5 mg/kg, injection twice a day (morning and evening)) (terbutalin hemisulfate; available from suppliers such as, for example, Sigma-Aldrich, catalog number T2528; diluted in a saline solution containing 0.9% NaCl) completely suppresses the mechanical allodynia after chronic treatment.

The graphs of FIG. 18 show that a treatment with the beta2-adrenergic agonist terbutalin (0.5 mg/kg, injection twice a day (morning and evening)) completely suppresses the mechanical allodynia of the right foot (foot with surgery bearing the “Cuff”) after chronic treatment without aspecifically affecting the response of the non-neuropathic left foot (foot without surgery).

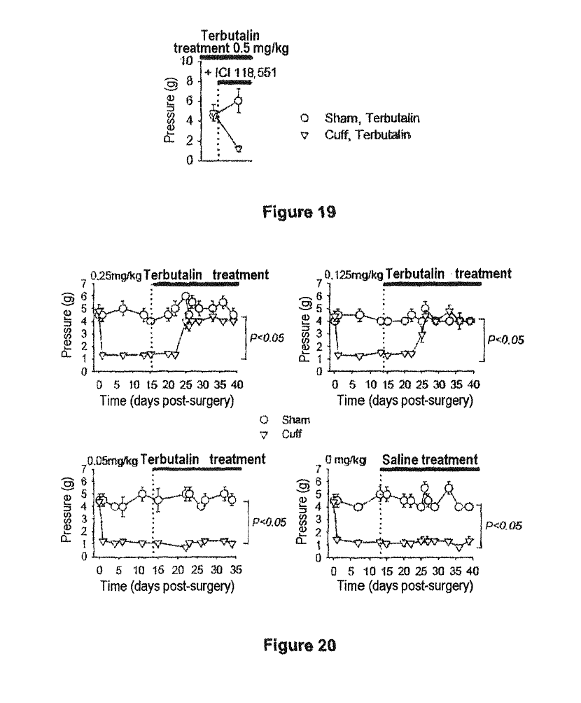

The graph of FIG. 19 shows that, after 3 weeks of treatment with terbutalin at the dose of 0.5 mg/kg (injection twice a day (morning and evening)), if terbutalin and the beta2-adrenoreceptor ICI 118,551 (“ICI”, 2 mg/kg, intraperitoneal, as described in example 5) are coadministered, this causes the allodynia to reappear in the neuropathic (“Cuff”) animals in a few days. The graph shows the nociceptive threshold of the animals after 3 weeks of treatment with terbutalin and after 4 days of cotreatment with ICI 118,551. The therapeutic effect of the terbutalin therefore clearly involves its action on beta2-adrenoreceptors.

The nociceptive sensitivity of the mice of the control (or “Sham”) group is not affected by the ICI 118,551 injections.

The graphs of FIG. 20 show that treatments with the beta2-adrenergic agonist terbutalin at the doses of 0.25 mg/kg or 0.125 mg/kg (injection twice a day (morning and evening)) completely suppress the mechanical allodynia of the right foot (neuropathic foot) after chronic treatment.

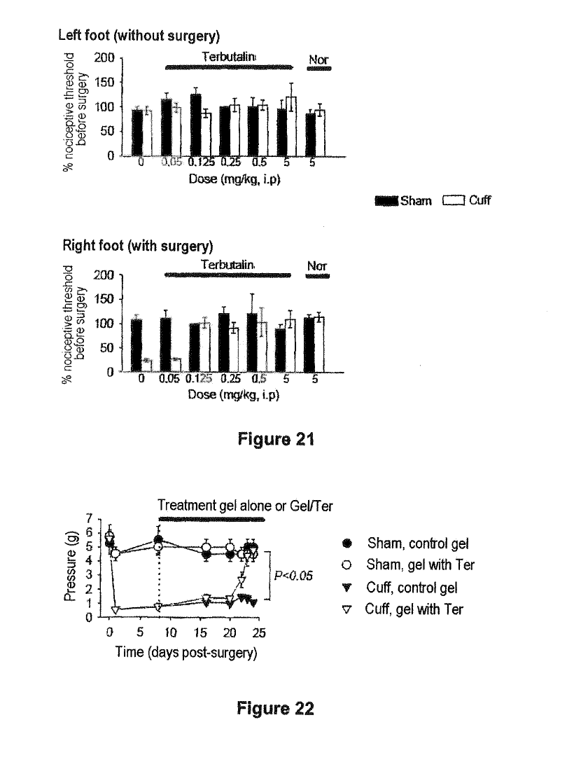

The graphs of FIG. 21 recapitulate the data on the dose-response study of the anti-allodynia action of terbutalin. They show the variation in nociceptive threshold as % of the sensitivity at the beginning of the experiment. The data are averaged over measurements made during the third week of treatment of the animals. The mechanical sensitivity of the animals treated with a placebo (saline solution, indicated as dose 0) remains stable during the experiment. The neuropathic (“Cuff”) animals experience a mechanical allodynia of the right foot which bears the cuff. The chronic treatment (3 weeks) of the neuropathic animals with the beta2-adrenergic agonist terbutalin at the doses of 0.125 mg/kg, 0.25 mg/kg, 0.5 mg/kg or 5 mg/kg suppresses the allodynia of the neuropathic right foot without affecting the mechanical sensitivity of the non-neuropathic left foot. These same treatments do not affect the control (“Sham”) animals. For comparison, the effect of the antidepressant nortriptylin (“Nor”, 5 mg/kg, twice a day (morning and evening)) is also presented. The antidepressant also does not impair the sensitivity of control animals. The model and the tests used are those described in example 1.

The nociceptive sensitivity of the mice of the control (or “Sham”) group is not affected by the terbutalin injections.

These data show for the first time that the administration of terbutalin can relieve neuropathic pain, in particular neuropathic allodynia.

The graph of FIG. 22 shows that a treatment with the beta2-adrenergic agonist terbutalin administered by application to the skin (0.1 ml of gel applied twice a day (morning and evening)) (2.5 mg/ml of terbutalin hemisulfate in a gel containing 5% of hydroxyethylcellulose diluted in 5% of ethanol and 95% water. Hydroxyethylcellulose available from suppliers such as, for example, Fluka, catalog number 54290) relieves the mechanical allodynia in the neuropathic (“Cuff”) mice after chronic treatment. The back of the animals was shaved so as to prevent the fur from inhibiting penetration of the gel. The gel is deposited on the back at the lumbar level while the animal is awake and the deposit is gently massaged until the product has penetrated. The control animals for the treatment follow the same procedure, but no beta2-adrenergic agonist was placed in the gel. The treatment with terbutalin began 8 days after the surgery for placing the cuffs around the sciatic nerve, the other experimental parameters being similar to those disclosed in example 1.

These data show for the first time that administration of terbutalin by application to the skin can relieve neuropathic pain, in particular neuropathic allodynia.

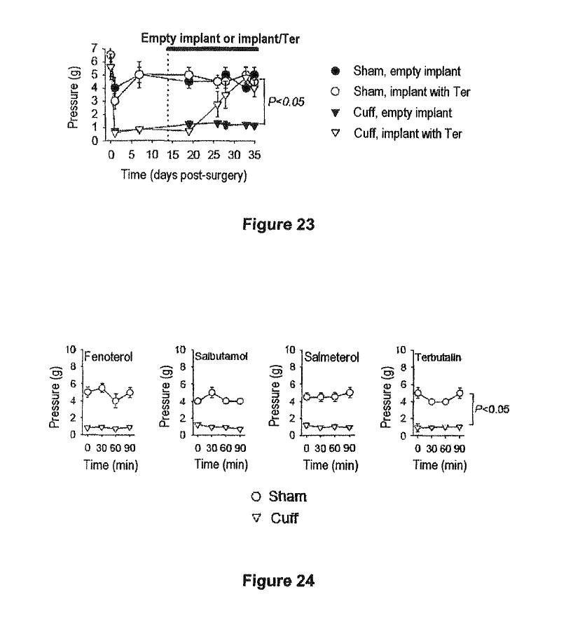

The graph of FIG. 23 shows that a treatment with the beta2-adrenergic agonist terbutalin delivered by inserting a subcutaneous implant (silicone tube implant, available from suppliers such as, for example, Sedat, catalog number 602 235) containing 10 mg of terbutalin hemisulfate, relieves the mechanical allodynia in the neuropathic (“Cuff”) mice. The implant makes it possible to model the continuous administration of modes of administration such as, for example, patches. The silicone tube, which has an internal diameter of 1.4732 mm and an external diameter of 1.9558 mm, is cut into sections 1.5 cm long. One of the ends is blocked with silicone adhesive (Silastic® Medical Adhesive Silicone Type A; available from suppliers such as, for example, Dow Corning, catalog number 891). After drying, the sections are each filled with 10 mg of terbutalin hemisulfate, the powder is tapped, and the section is cut again to a final length of approximately 1 cm and is blocked up with the silicone adhesive. The implant is implanted into the mouse under halothane gas anesthesia. Under anesthesia, the nape of the neck and the top of the back of the animal are shaved, an incision is made in the top of the nape of the neck and the implant is slid in subcutaneously using a cannula guide. The control animals for the treatment undergo the same procedure, with an empty implant being inserted. The implants were inserted 14 days after the surgery for placing the cuffs around the sciatic nerve as described in example 1.

These data show for the first time that continuous administration of terbutalin can relieve neuropathic pain, in particular neuropathic allodynia.

### Example 15

### Absence of Effect of the Acute Injection (First Injection) of the Beta2-Adrenoreceptor Agonists Fenoterol, Salbutamol, Salmeterol and Terbutalin

The graph of FIG. 24 shows that the first injection of the beta2-adrenergic agonists fenoterol (0.7 mg/kg, intraperitoneal), salbutamol (2 mg/kg, intraperitoneal), salmeterol (1 mg/kg, intraperitoneal) or terbutalin (5 mg/kg, intraperitoneal), as described in the examples above, has no effect on the mechanical allodynia of the neuropathic (“Cuff”) mice for 2 weeks.

The mice of the control (or “Sham”) group are also unaffected by the first administration of these agonists.

### Example 16

### THERAPY: Recapitulation, Effect of the Beta-Adrenergic Agonists in the Neuropathic Animals

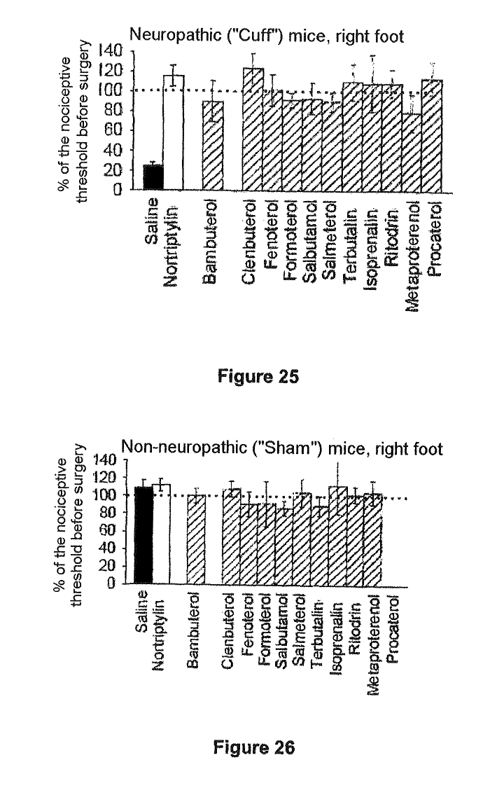

The graph of FIG. 25 presents the variation in nociceptive threshold as % of the sensitivity at the beginning of the experiment. The data are averaged over measurements made during the third week of treatment of the animals.

The neuropathic (“Cuff”) animals treated with a placebo (saline solution) maintain the large decrease in their nociceptive threshold, which illustrates the presence of allodynia (pain).

The chronic treatment (14-21 days) of the neuropathic animals with the beta-adrenergic agonists bambuterol, clenbuterol, fenoterol, formoterol (0.5 mg/kg), salbutamol, salmeterol, terbutalin (5 mg/kg), isoprenalin, ritodrin, metaproterenol or procaterol, as described in the examples above, all relieve this allodynia.

For comparison, the effect of the antidepressant nortriptylin is also presented. The treatment doses and modes are those described above in examples 2, 7, 9 to 14 and 20. The model and the tests used are those described in example 1.

The data presented here demonstrate for the first time that a chronic treatment with beta-adrenergic or beta2-adrenergic agonists administered alone makes it possible to relieve neuropathic allodynia.

### Example 17

### Recapitulation, Effect of the Beta-Adrenergic Agonists in Control Animals

The graph of FIG. 26 presents the variation in nociceptive threshold as % of the sensitivity at the beginning of the experiment. The data are averaged over measurements made during the third week of treatment of the animals.

The control (“Cuff”) animals are treated with a placebo (saline solution). Their mechanical sensitivity remains stable during the experiment.

The chronic treatment (14-21 days) of the neuropathic animals with the beta-adrenergic agonists bambuterol, clenbuterol, fenoterol, formoterol (0.5 mg/kg), salbutamol, salmeterol, terbutalin (5 mg/kg), isoprenalin, ritodrin, metaproterenol or procaterol, do not affect the control animals. For comparison, the effect of the antidepressant nortriptylin is also presented. The antidepressant also does not impair the sensitivity of control animals. The treatment doses and modes are those described above in examples 2, 7, 9 to 14 and 20. The model and the tests used are those described in example 1.

These data show that a chronic treatment with beta-adrenergic or beta2-adrenergic agonists administered alone does not affect the sensitivity of control animals.

### Example 18

### Effect of Blocking Beta2-Adrenoreceptors on the Action of Bambuterol, of Fenoterol, of Formoterol and of Salmeterol

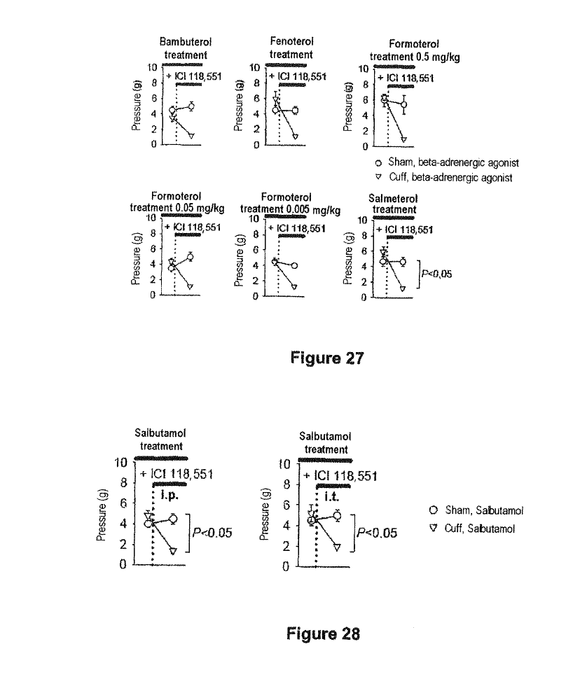

The graphs of FIG. 27 show that, after 3 weeks of treatment with the beta-adrenergic agonists bambuterol, fenoterol, formoterol or salmeterol, if these agonists and the beta2-adrenoreceptor antagonist ICI 118,551 (“ICI”, 2 mg/kg, intraperitoneal) are coadministered, this causes the allodynia to reappear in the neuropathic (“Cuff”) mice in a few days. The graphs show the nociceptive threshold of the animals after 3 weeks of treatment with the agonists and after 3 to 6 days of cotreatment with ICI 118,551. The treatment doses and modes are those described above in examples 5, 9 to 11 and 13. The model and the tests used are those described in example 1.

The therapeutic effect of these agonists therefore clearly involves their action on beta2-adrenoreceptors.

The nociceptive sensitivity of the mice of the control (or “Sham”) group is not affected by the ICI 118,551 injections.

### Example 19

### Effect of Blocking Beta2-Adrenoreceptors on the Action of Salbutamol

The graphs of FIG. 28 show that, after 3 weeks of treatment with the beta2-adrenergic agonist salbutamol, if this agonist and the beta2-adrenoreceptor antagonist ICI 118,551 are coadministered, this causes the allodynia to reappear in the neuropathic (“Cuff”) mice in a few days. The left-hand graph shows the nociceptive threshold of the animals after 3 weeks of treatment with salbutamol and after 3 days of cotreatment with ICI 118,551 coinjected intraperitoneally (2 mg/kg). The right-hand graph shows the nociceptive threshold of the animals after at least 3 weeks of treatment with salbutamol and after 2 injections of cotreatment with ICI 118,551 administered intrathecally before each injection of salbutamol (2 mg/kg, intraperitoneal). The treatment doses and modes are those described above in examples 5 and 12 for the intraperitoneal injections and below for the description of the intrathecal injection procedure. The model and the tests used are those described in example 1. The therapeutic effect of salbutamol therefore clearly involves its action on beta2-adrenoreceptors.

The results of the intrathecal injection show that the spinal beta2-adrenoreceptors and/or those of the dorsal root ganglia are important for the therapeutic effect of salbutamol.

The procedure for intrathecal (i.t.) injection of the beta2-adrenoreceptor antagonist ICI 118,551 (3 μg in 10 μl) is carried out under halothane gas anesthesia according to the protocol described by Inoue et al. (Nature Medicine 2004, 10:712-718 (12)). These i.t. injections are carried out just before the intraperitoneal injection of salbutamol (2 mg/kg, i.p.). A 27-gauge injection needle connected to a 50 μl Hamilton syringe is inserted, between the L₅ and L₆ vertebrae, into the subarachnoid space. The correct placement of the needle is verified by the presence of a reflex movement of the tail of the mouse. The mice are pretested before this cotreatment procedure, and then cotreated twice (a morning and the corresponding evening) before being retested the following morning for their mechanical nociceptive sensitivity.

The nociceptive sensitivity of the mice of the control (or “Sham”) group is not affected by the ICI 118,551 injections, whether the latter are intraperitoneal or intrathecal.

### Example 20

### Effect of a Stimulation of Beta- or Beta2-Adrenoreceptors: other Tests

Under the conditions described in the examples above, the following molecules are tested:
bitolterol (for example, between 0.05 and 5 mg/kg, twice a day, intraperitoneal);
isoprenalin or isoproterenol (for example, between 0.05 and 5 mg/kg, twice a day, intraperitoneal);
levalbuterol (for example, between 0.05 and 5 mg/kg, twice a day, intraperitoneal);
metaproterenol (for example, between 0.05 and 5 mg/kg, twice a day, intraperitoneal);
pirbuterol (for example, between 0.25 and 50 mg/kg, twice a day, intraperitoneal);
procaterol (for example, between 0.05 and 5 mg/kg, twice a day, intraperitoneal);
reproterol (for example, between 0.05 and 5 mg/kg, twice a day, intraperitoneal);
ritodrin (for example, between 0.25 and 50 mg/kg, twice a day, intraperitoneal;
tulobuterol (for example, between 0.05 and 5 mg/kg, twice a day, intraperitoneal).

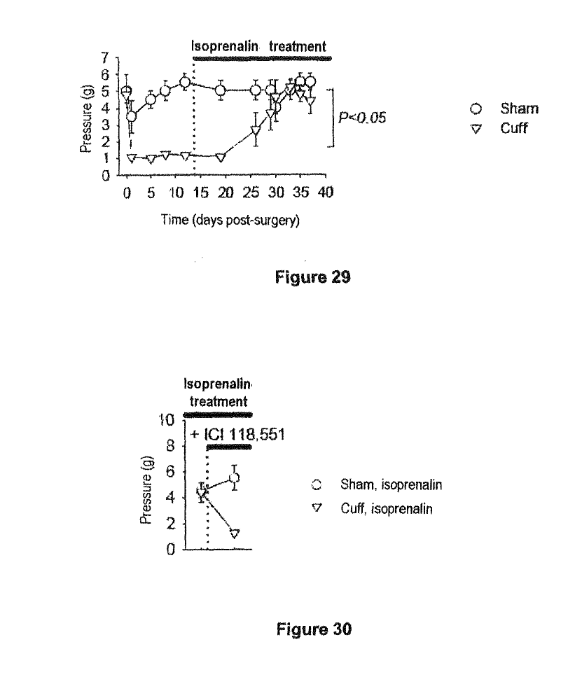

The graph of FIG. 29 shows that a treatment with the beta-adrenergic agonist isoprenalin (0.5 mg/kg, injection twice a day (morning and evening)) (DL-isoproterenol chloride; available from suppliers such as, for example, Sigma-Aldrich, catalog number I5627; diluted in a saline solution containing 0.9% NaCl) completely suppresses the mechanical allodynia in the neuropathic (“Cuff”) mice after chronic treatment.

These data show for the first time that the administration of isoprenalin can relieve neuropathic pain, in particular neuropathic allodynia.

The graph of FIG. 30 shows that, after 3 weeks of treatment with the beta-adrenergic agonist isoprenalin, if the beta2-adrenoreceptor antagonist ICI 118,551 (“ICI”, 2 mg/kg, intraperitoneal) is coadministered, this causes the allodynia to reappear in the neuropathic (“Cuff”) mice in a few days. The graph shows the nociceptive threshold of the animals after 3 weeks of treatment with isoprenalin and after 4 days of cotreatment with ICI 118,551. The model and the tests used are those described in example 1.

The therapeutic effect of this agonist therefore clearly involves its action on beta2-adrenoreceptors.

The nociceptive sensitivity of the mice of the control (or “sham”) group is not affected by the ICI 118,551 injections.

The graph of FIG. 31 shows that a treatment with the beta2-adrenergic agonist ritodrin (10 mg/kg, injection twice a day (morning and evening)) (ritodrin hydrochloride; available from suppliers such as, for example, Sigma-Aldrich, catalog number R0758; diluted in a saline solution containing 0.9% NaCl and containing 0.3% ascorbic acid antioxidant) completely suppresses the mechanical allodynia in the neuropathic (“Cuff”) mice after chronic treatment.

These data show for the first time that the administration of ritodrin can relieve neuropathic pain, in particular neuropathic allodynia.

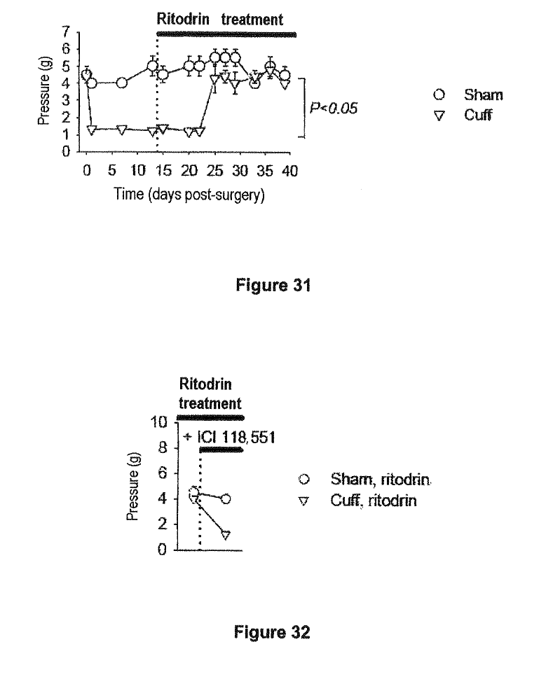

The graph of FIG. 32 shows that, after 3 weeks of treatment with the beta-adrenergic agonist ritodrin, if the beta2-adrenoreceptor antagonist ICI 118,551 (“ICI”, 2 mg/kg, intraperitoneal) is coadministered, this causes the allodynia to reappear in the neuropathic (“Cuff”) mice in a few days. The graph shows the nociceptive threshold of the animals after 3 weeks of treatment with ritodrin and after 4 days of cotreatment with ICI 118,551. The model and the tests used are those described in example 1.

The therapeutic effect of this agonist therefore clearly involves its action on beta2-adrenoreceptors.

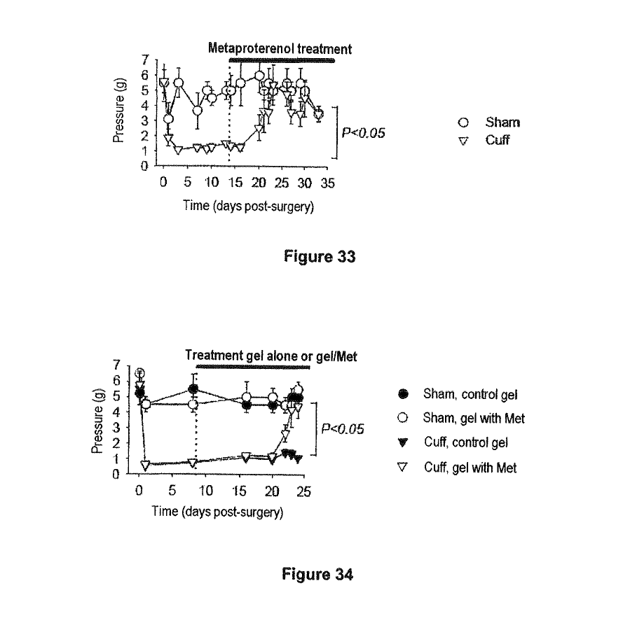

The graph of FIG. 33 shows that a treatment with the beta2-adrenenergic agonist metaproterenol (1 mg/kg, injection twice a day (morning and evening)) (metaproterenol hemisulfate; available from suppliers such as, for example, Sigma-Aldrich, catalog number M2398; diluted in a saline solution containing 0.9% NaCl) completely suppresses the mechanical allodynia in the neuropathic (“Cuff”) mice after chronic treatment.

These data show for the first time that the administration of metaproterenol can relieve neuropathic pain, in particular neuropathic allodynia.

The graph of FIG. 34 shows that a treatment with the beta2-adrenergic agonist metaproterenol administered by application to the skin (0.1 ml of gel applied twice a day (morning and evening)) (1 mg/ml of metaproterenol hemisulfate in a gel containing 5% of hydroxyethylcellulose diluted in 5% of ethanol and 95% water. Hydroxyethylcellulose available from suppliers such as, for example, Fluka, catalog number 54290) relieves the mechanical allodynia in the neuropathic (“Cuff”) mice after chronic treatment. The back of the animals was shaved so as to prevent the fur from inhibiting penetration of the gel. The gel is deposited onto the back at the lumbar level while the animal is awake and the deposit is gently massaged until the product has penetrated. The control animals for the treatment follow the same procedure, but no beta2-adrenergic agonist was included in the gel. The treatment with metaproterenol began 8 days after the surgery for placing the cuffs around the sciatic nerve, and the other experimental parameters are similar to those described in example 1.

These data show for the first time that administration of metaproterenol by application to the skin can relieve neuropathic allodynia.

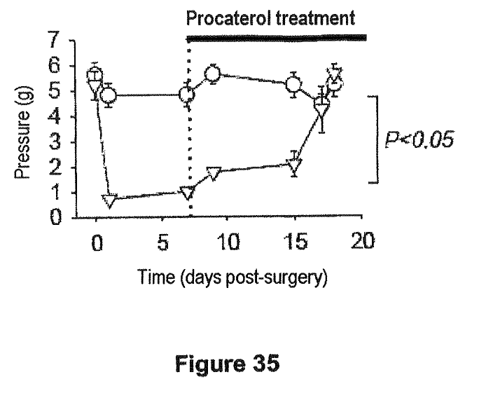

The graph of FIG. 35 shows that a treatment with the beta2-adrenergic agonist procaterol (0.8 mg/kg, injection twice a day (morning and evening)) (procaterol hydrochloride; available from suppliers such as, for example, Biotrend AG, catalog number BN0432; diluted in a saline solution containing 0.9% NaCl) completely suppresses the mechanical allodynia in the neuropathic (“Cuff”) mice after chronic treatment.

These data show for the first time that the administration of procaterol can relieve neuropathic pain, in particular neuropathic allodynia.

### REFERENCE LIST

(1) Bouhassira D, Lantéri-Minet M, Attal N, Laurent B, Touboul C. Prevalence of chronic pain with neuropathic characteristics in the general population. Pain 2008, 136:380-387.
(2) Merksey H, Bogduk N, editors. Classification of Chronic Pain. IASP Press, Seattle 1994.
(3) McQuay H J, Tramér M, Nye B A, Caroll D, Wiffen P J, Moore R A. A systemic review of antidepressants in neuropathic pain. Pain 1996, 68:217-227.
(4) Hempenstall K, Nurmikko T J, Johnson R W, A'Hern R P, Rice A S C. Analgesic therapy in postherpetic neuralgia: a quantitative systematic review. PLoS Medicine 2:e164.
(5) Gilron I, Watson C P, Cahill C M, Moulin D E. Neuropathic pain: a practical guide for the clinician. CMAJ 2006, 175:265-275.
(6) Attal N, Cruccu G, Haanpaa M, Hansson P, Jensen T S, Nurmikko T, Sampaio C, Wiffen P, EFNS Task Force. European Journal of Neurology 2006, 13:1153-1169.
(7) Moulin D E, Clark A J, Gilron I, Ware M A, Watson C P, Sessle B J, Coderre T, Morley-Foster P K, Stinson J, Boulanger A, Peng P, Finley G A, Taenzer P, Squire P, Dion D, Cholkan A, Gilani A, Gordon A, Henry J, Jovey R, Lynch M, Mailis-Gagnon A, Panju A, Rollman G B, Velly A, Canadian Pain Society. Pharmacological management of chronic neuropathic pain—consensus and guidelines from the Canadian Pain Society. Pain Research Management 2007, 12:13-21.
(8) Dworkin R H, O'Connor A B, Backonja M, Farrar J T, Finerup N B, Jensen T S, Kalso E A, Loeser J D, Miaskowski C, Nurmikko T J, Portenoy R K, Rice A S C, Stacey B R, Treede R D, Turk D C, Wallace M S. Pharmacological management of neuropathic pain: evidence-based recommendation. Pain 2007, 132:237-251.
(9) Crassous P A, Denis C, Paris H, Sénard J M. Interest of alpha2-adrenergic agonists and antagonists in clinical practice: background, facts and perspectives. Current Topics in Medicinal Chemistry 2007, 7:187-194.
(10) Benbouzid M, Gavériaux-Ruff C, Yalcin I, Waltisperger E, Tessier L H, Muller A, Kieffer B L, Freund-Mercier M J, Barrot M. Delta-opioid receptors are critical for tricyclic antidepressant treatment of neuropathic allodynia. Biological Psychiatry 2008, 63:633-636.
(11) Benbouzid M, Pallage V, Rajalu M, Waltisperger E, Doridot S, Poisbeau P, Freund-Mercier M J, Barrot M. Sciatic nerve cuffing in mice: a model of sustained neuropathic pain. European Journal of Pain 2008, 12:591-599.
(12) Inoue M, Rashid M H, Fujita R, Contos J J, Chun J, Ueda H. Initiation of neuropathic pain requires lysophosphatidic acid receptor signalling. Nature Medicine 2004; 10:712-718.

1. A method of treating neuropathic allodynia comprising administering a therapeutically effective amount of a beta-adrenergic agonist as an active ingredient of a drug.

2. The method as claimed in claim 1, in which the neuropathic allodynia is chronic neuropathic allodynia.

3. The method as claimed in claim 1 or 2, in which the beta-adrenergic agonist is chosen from the group comprising bambuterol, bitolterol, clenbuterol, fenoterol, formoterol, isoproterenol, levalbuterol, metaproterenol, pirbuterol, procaterol, reproterol, ritodrin, salbutamol, salmeterol, terbutalin and tulobuterol.

4. The method as claimed in claim 1, in which the beta-adrenergic agonist is administered chronically.

5. The method as claimed in claim 1, in which the beta-adrenergic agonist is one, two or three times a day.

6. The method as claimed in claim 1, in which the beta-adrenergic agonist is administered at a dose of between 0.01 and 20 mg/day.

7. The method as claimed in claim 1, in which the beta-adrenergic agonist is administered at a dose of between 0.05 and 15 mg/day.

8. The method as claimed in claim 1, in which the beta-adrenergic agonist is administered at a dose of between 0.05 and 10 mg/day.

9. The method as claimed in claim 1, in which the beta-adrenergic agonist is administered at a dose of between 0.1 and 5 mg/day.

10. The method as claimed in claim 1, in which the drug is for human use.

11. The method as claimed in claim 1, in which the drug is for veterinary use.

12. The method as claimed in claim 1, in which the drug is in a form chosen from the group comprising an injectable form, a syrup, an oral solution, a tablet, a breakable tablet, a film-coated tablet, a breakable film-coated tablet, a gastroresistant tablet, a coated tablet, a dispersible tablet, a chewable tablet, a blister pack, a gel capsule, an effervescent product, a breakable effervescent tablet, a powder for oral solution, granules for oral suspension, a suspension for inhalation, a powder for inhalation, a solution for inhalation by nebulizer, a powder for inhalation in a gel capsule, a suppository, an eye lotion, a cream, an ointment, a gel, a spray, a patch, a lozenge, and a solution for intravenous perfusion, or for peridural, intra- or peri-articular, peripheral, regional, paravertebral or intrathecal administration, or infiltration.
